(19) United States
(12) Plant Patent Application Publication
(10) Pub. No.: US 2019/0327867 P1
(43) Pub. Date: Oct. 24, 2019

(54) Strawberry Plant Named 'NSG 120'

(50) ;

(71) Applicant: Nova Siri Genetics S.r.l., Nova Siri (MT) (IT)

(72) Inventor: Nicola Tufaro, Nova Siri (MT) (IT)

(73) Assignee: Nova Siri Genetics S.r.l., Nova Siri (MT) (IT)

(21) Appl. No.: 16/501,377

(22) Filed: Apr. 3, 2019

(30) Foreign Application Priority Data
Apr. 23, 2018 (EM) 2018/1111

(51) Int. Cl. A01H 6/74

(52) U.S. Cl. PLT/208

(57) ABSTRACT

A new and distinct variety of strawberry plant, herein referred to by its cultivar name, ‘NSG 120’, is provided which forms in abundance attractive early-ripening large, medium red primarily conical fruit having firm flesh that is longer than broad in configuration. The growth habit is semi-dense, semi-upright. White inflorescence is displayed. Commonly a calyx is displayed which is larger than the diameter of the corolla when open. The new variety is particularly well suited for the commercial industry.

Botanical/commercial classification: Latin name—Fragaria x ananassa Duchesne. Common name—Strawberry Plant.

Varietal denomination: ‘NSG 120’.

### CROSS-REFERNCE TO RELATED APPLICATIONS

This application claims priority to Community Plant Variety Rights Application Number 2018/1111 which was filed in European Union through Community Plant Variety Office on Apr. 23, 2018, of which the content of is hereby expressly incorporated by reference in its entirety for all purposes.

### SUMMARY OF THE INVENTION

The new and distinct short-day strawberry plant variety of the present invention was the product of a controlled breeding program that was carried out at Nova Siri (MT) Italy located at 40°08′40″ N-16°39′40″ E and 10 meters above sea level, wherein two parents were crossed which previously had been studied in the hope that they would contribute the desired characteristics. The female parent (i.e., the seed parent) was the CHARLENE variety (U.S. Plant Pat. No. 28,220 P3 and European Union Community Plant Variety Rights No. 45319), which is a strawberry variety plant with medium-season yield and produces fruit with high contents of sugar. The male parent (i.e., pollen parent) was the MELISSA variety (U.S. Plant Pat. No. 28081P3 and European Union Community Plant Variety Rights No. 45318), which is a strawberry variety with high early-season yield, produces very high yield and very large primary and secondary fruits.

The parentage can be summarized as follows:

‘CHARLENE’ x ‘MELISSA’.

The seeds resulting from the above pollination were sown and small plants were obtained which were physically different from each other. Selective study and testing resulted in the identification of a single short-day strawberry plant of the new variety.

It was found that the new strawberry plant of the present invention possesses the following combination of characteristics:
(a) exhibits a semi-dense, semi-upright growth habit,
(b) displays, on an early basis, white inflorescence at a level generally above the foliage,
(c) commonly displays a calyx that is larger than the diameter of the corolla when open, and
(d) forms in abundance attractive early-ripening large, medium red primarily conical fruit having firm flesh that is longer than broad in configuration.

The new variety well meets the needs of the horticultural industry. The new variety possesses characteristics that commonly are sought by commercial strawberry growers. For example, the new variety provides uniform attractive firm medium red early-ripening fruit in good yields. Accordingly, the new variety is considered promising for commercial introduction. The new variety requires an induction period for flowering. This may be achieved by growing in a colder climate away from the equator or at a higher altitude above sea level.

The new variety can be readily distinguished from its ancestors. More specifically, the ‘CHARLENE’ variety (i.e., the seed parent) displays generally conical shape fruit which is darker in external and internal color compared to the conical, slightly rhomboid, shaped fruit of the new variety. Specifically, ‘CHARLENE’ displays fruit with an external color commonly near Red Group 45B to Red Group 46A and an internal color commonly near Orange-Red Group 34A to Orange-Red Group 34B, whereas the new variety displays fruit with an external color commonly near Red Group 43A to Red Group 43B and an internal color commonly near Orange-Red Group 33A to Orange-Red Group 33B. Additionally, the ‘MELISSA’ variety (i.e., the pollen parent) provides generally rhomboid shape fruit with an external color and internal core color which is darker compared to the conical, slightly rhomboid, shape fruit of the new variety. Specially, ‘MELISSA’ provides fruit with an external color commonly near Red Group 45B to Red Group 46B and an internal color commonly near Orange-Red Group 34A to Orange-Red Group 34B, whereas the new variety displays fruit with an external color commonly near Red Group 43A to Red Group 43B and an internal color commonly near Orange-Red Group 33A to Orange-Red Group 33B. Moreover, the new variety can be readily distinguished from non-parental related similar varieties. For example, the ‘NSG 1117’ variety (European Union Community Plant Variety Rights Application No. 2016/1694 and not patented in the U.S.) provides fruit that is darker in color compared to the fruit of the new variety and the time between first blooming and first fruit ripening for the ‘NSG 1117’ variety is longer compared to the new variety. Specifically, ‘NSG 1117’ provides fruit with an external color commonly near Red Group 45A and an internal color commonly near Red Group 42A, whereas the new variety is provides fruit with an external color commonly near Red Group 43A to Red Group 43B and an internal color commonly near Orange-Red Group 33A to Orange Red Group 33B.

The new variety has been found to undergo asexual propagation in Poland and Italy by a number of routes, including by use of stolons and in vitro tissue culture. Specifically, the new variety has been asexually reproduced in a nursery setting by the use of stolons at Zielona Gora-Ochla, Poland located at 51°848 N-15°447 E and at Nova Siri (MT), Italy by in vitro tissue culture. No rotting problems were observed on the roots during cultivation. The combination of characteristics exhibited by the new variety has been found to be stable and reliably transmitted to succeeding generations following such asexual reproduction. Accordingly, the new variety undergoes asexual propagation in a true-to-type manner by such asexual reproduction.

The new variety has been named ‘NSG 120’. The new plant variety initially was designated as EE 14 P3 15 120.

### BRIEF DESCRIPTION OF THE PHOTOGRAPHS

The accompanying photographs show, as nearly true as it is reasonably possible to make the same in color illustrations of this character, typical specimens of the new variety. The illustrated strawberry plants of the new variety were asexually reproduced by stolons in a nursery at Zielona Gora-Ochla, Poland at 51°848 N-15°447 E and were planted under the cover of plastic tunnels during mid-October 2017 at Nova Siri (MT), Italy located at 40°08′40″ N-16°39′40″ E at 10 meters above sea level.

FIG. 1—illustrates rows of early fruiting plants on Jan. 18, 2018, wherein the newly formed flowers are commonly disposed above the foliage and first early conical large red fruits are present.

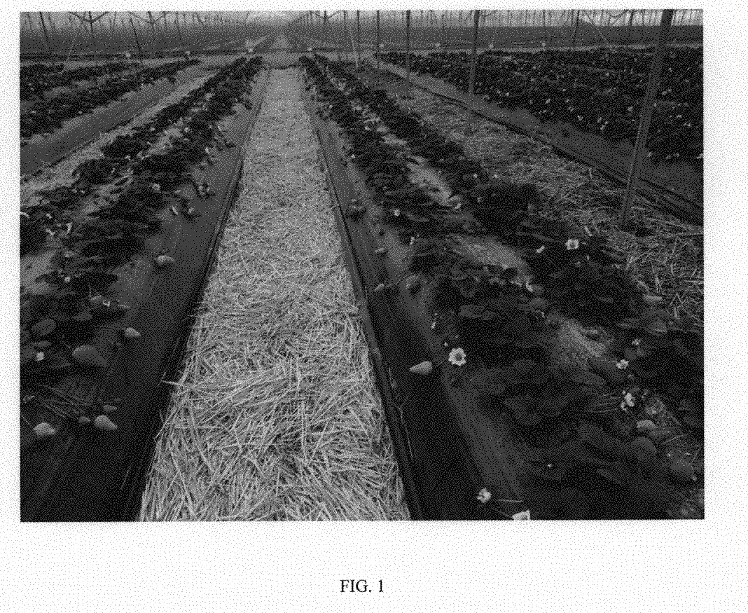

FIG. 2—illustrates two semi-upright fruiting plants on Feb. 8, 2018, wherein abundant early red fruit production is apparent.

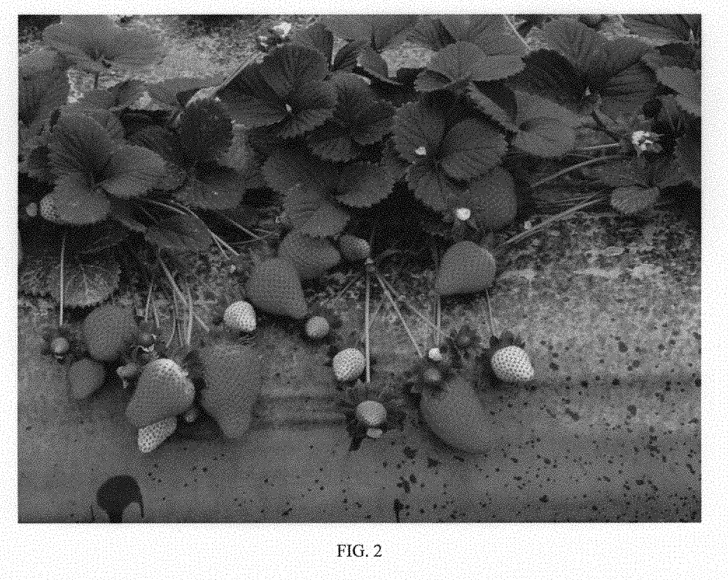

FIG. 3—illustrates a row of fruiting plants on Mar. 15, 2018, wherein medium vigor plants with semi-upright and semi-dense vegetation, newly formed flowers, uniform red fruits, and long peduncles are apparent.

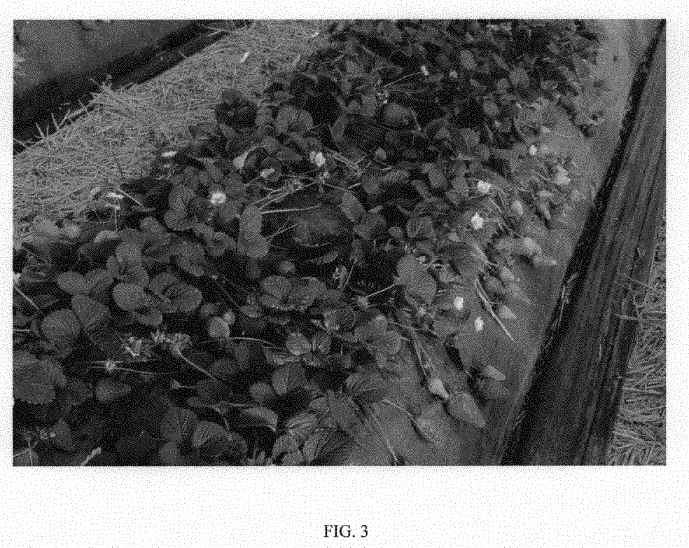

FIG. 4—illustrates a row of fruiting plants on Apr. 20, 2018, wherein abundant flowers and fruits in all phases of development are present.

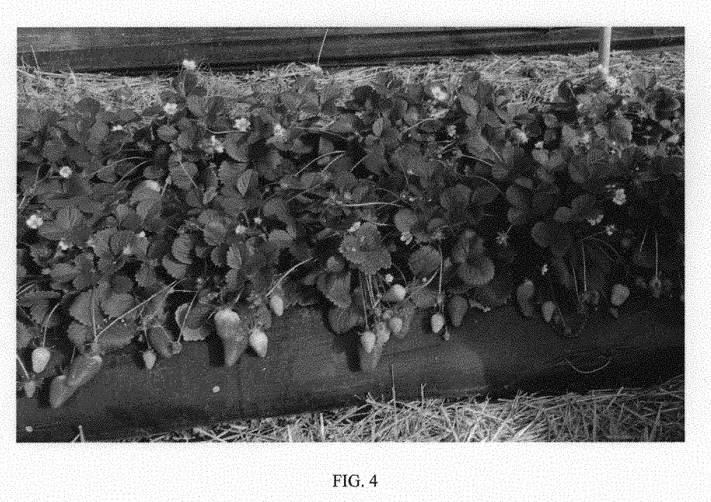

FIG. 5—illustrates specimens of three-leaflet leaves—upper surface, wherein the petiole and stipules are further apparent (designate in image as EE14.P3.15.120). Dimensions in centimeters and inches are included.

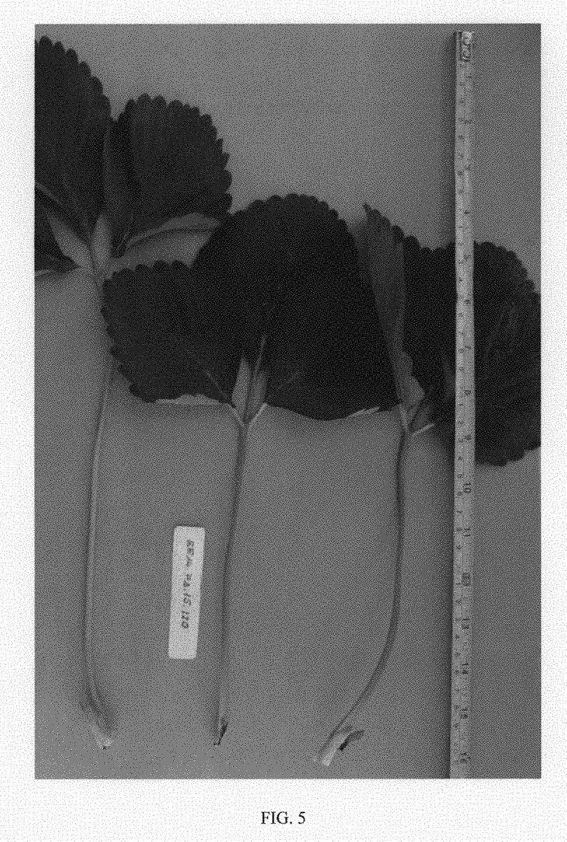

FIG. 6—illustrates a specimen of a typical three-leaflet leaf—upper surface (designate in image as EE14.P3.15.120). Dimensions in centimeters and inches are included.

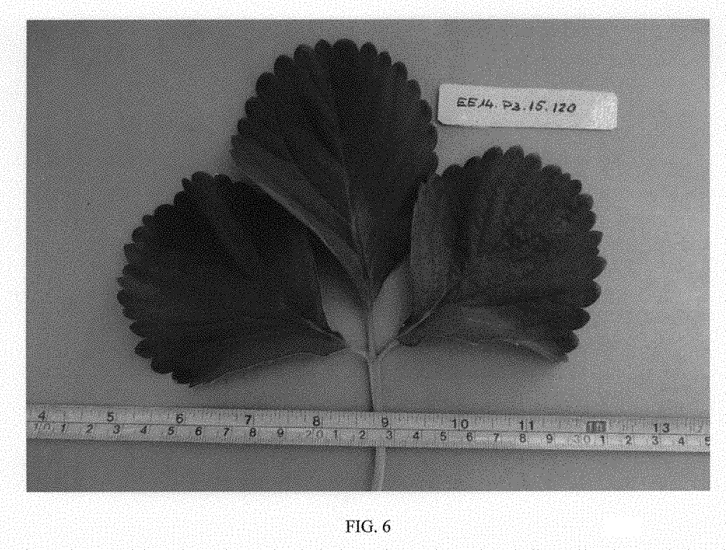

FIG. 7—illustrates a specimen of a typical three-leaflet leaf—under surface (designate in image as EE14.P3.15.120). Dimensions in centimeters and inches are included.

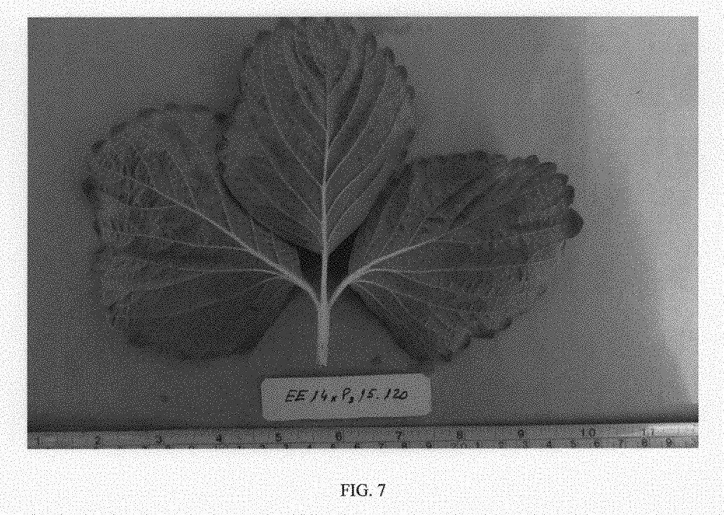

FIG. 8—illustrates a specimen of an inflorescence (designate in image as EE14.P3.15.120). Dimensions in centimeters and inches are included.

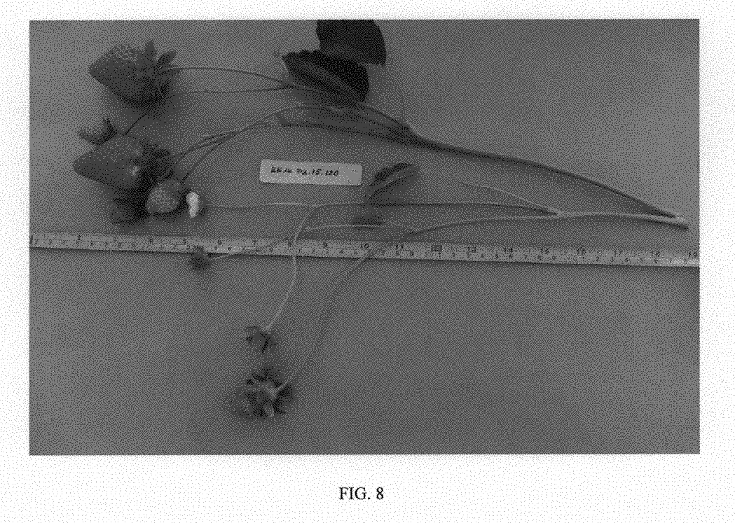

FIG. 9—illustrates specimens of corolla—close view (designate in image as EE14.P3.15.120). Dimensions in centimeters and inches are included.

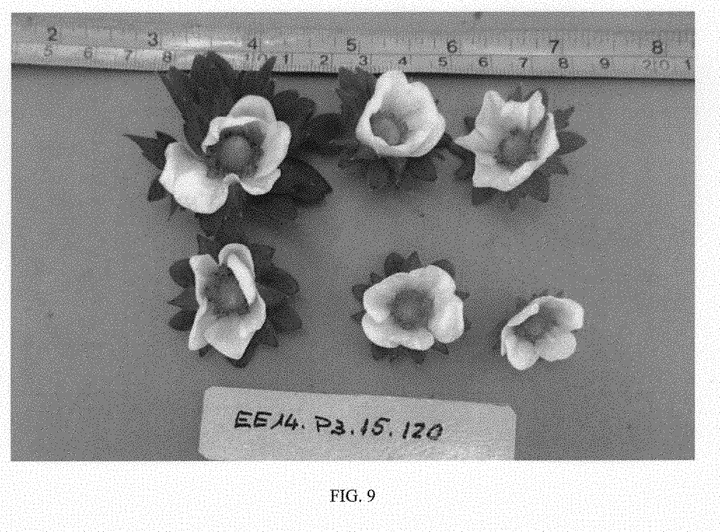

FIG. 10—illustrates specimens of calyx—close view (designate in image as EE14.P3.15.120). Dimensions in centimeters and inches are included.

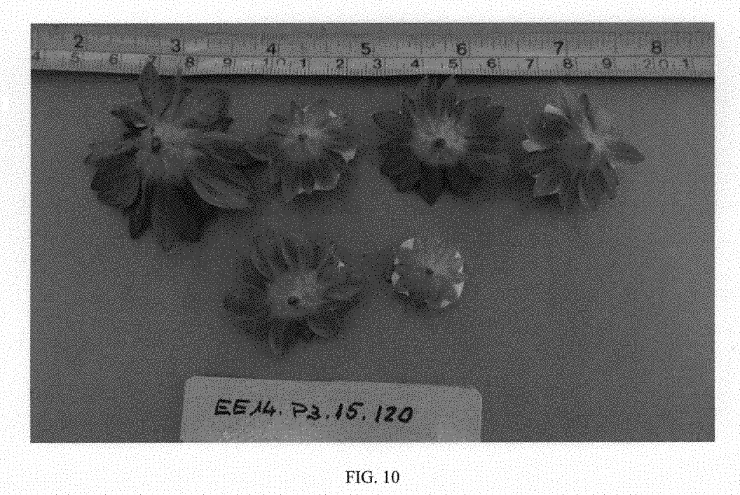

FIG. 11—illustrates specimens of petals of the flowers (designate in image as EE14.P3.15.120). Dimensions in centimeters and inches are included.

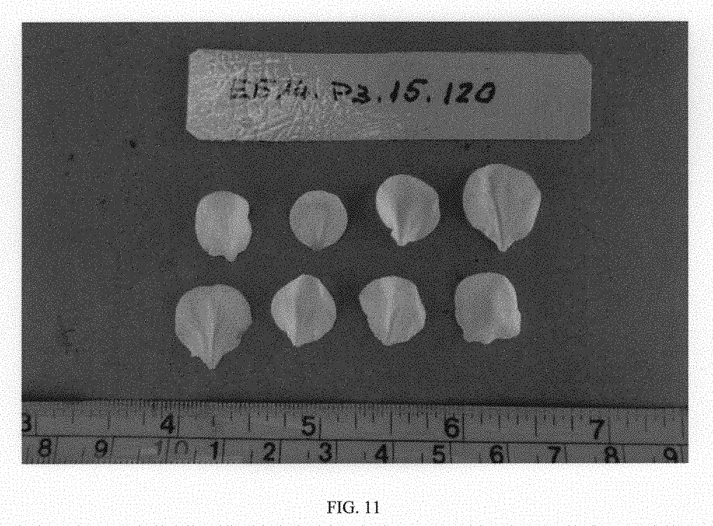

FIG. 12—illustrates specimens of mature fruit—whole (designate in image as EE14.P3.15.120). Dimensions in centimeters and inches are included.

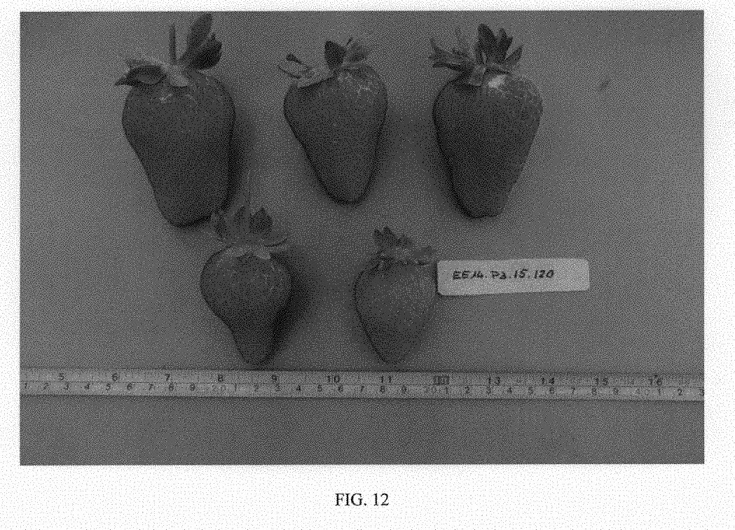

FIG. 13—illustrates specimens of mature fruit—internal sections (designate in image as EE14.P3.15.120). Dimensions in centimeters and inches are included.

FIG. 14—illustrates specimen of runners (stolons).

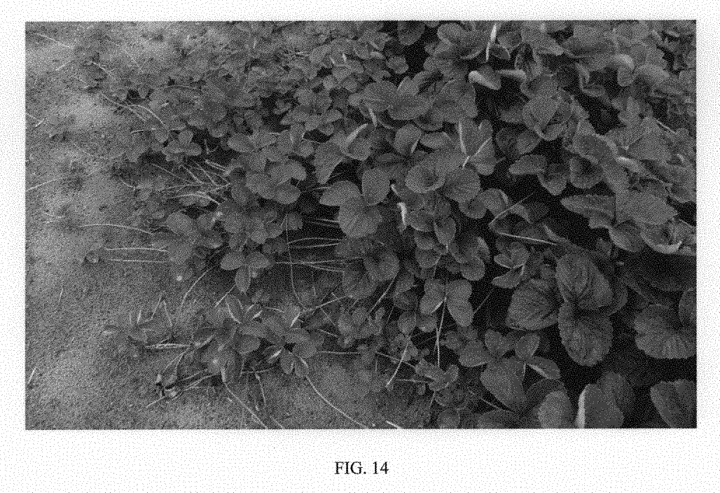

FIG. 15—illustrates specimens of stipule and petiole (designate in image as EE14.P3.15.120). Dimensions in centimeters and inches are included.

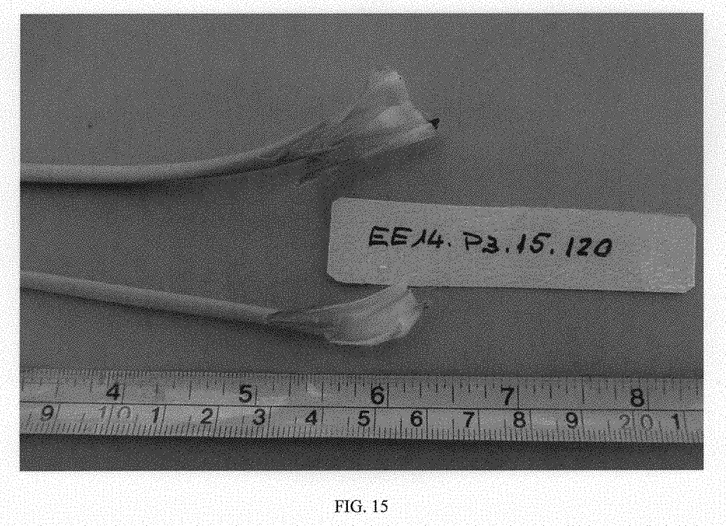

### DETAILED BOTANICAL DESCRIPTION

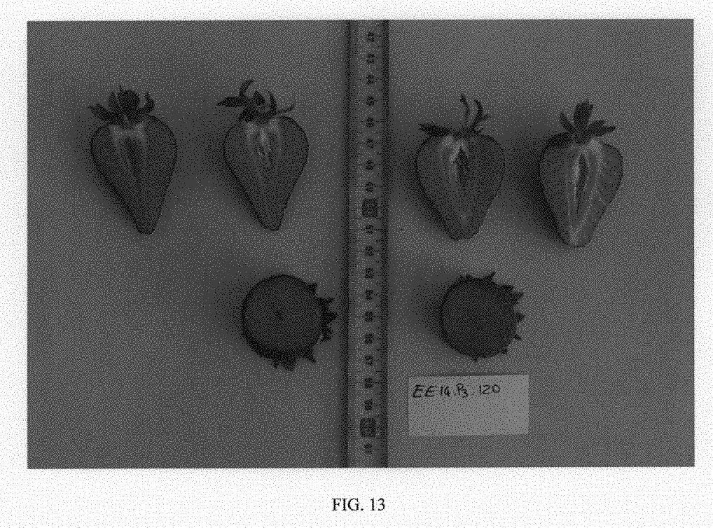

The chart used in the identification of colors is that of The Royal Horticultural Society (“R.H.S.” Colour Chart), London, England, Edition V. The terminology which precedes reference to the chart has been added to indicate the corresponding color in more common terms. The description is based on plants which were reproduced asexually by the use of stolons at Zielona Gora-Ochla, Poland located at 51°848 N-15°447 E and growing under the cover of plastic tunnels at Nova Siri (MT), Italy located at 40°08′40″ N-16°39′40″ E and 10 meters above sea level.
Plant:
   Type.—Short-day.
   Configuration.—Semi-upright and semi-dense.
   Vigor.—Medium to weak.
   Leaf.—Approximately 16 cm to 19 cm in size.
   Leaflets.—Number commonly 3. — size: medium-to large in size, approximately 10.5 cm in length on average, and approximately 8 cm in width on average. — terminal leaflet: commonly moderately longer in length than width, possesses a generally concave cross-section, possesses a crenate margin, and an acute base. — blistering: commonly is weak in quantity. — glossiness on the upper surface: weak. — color: variegated coloration commonly is absent; upper surface coloration commonly being near Green Group N141A to Green Group N141B; and under surface commonly being near Green Group 138C to Green Group 138D.
   Stolons.—High in quantity, medium pubescence in density, color is commonly near Yellow-Green Group 145B to Yellow-Green Group 145C, weak or absent anthocyanin coloration, and the distance between plants rooted in the same stolon is approximately 27 cm to 40 cm on average.
   Petioles.—Approximately 17 cm to 25 cm in length on average, color is commonly near Yellow-Green Group N144C, and commonly bear generally horizontally disposed fine pubescence.
   Stipules.—Approximately 2.5 cm to 3 cm in length on average, and commonly bear some weak anthocyanin coloration of near Red-Purple Group 61C to Red-Purple Group 61D.
Inflorescence:
   Flowering time.—Very early.
   Flower disposition.—Generally above the foliage.
   Flower number.—Medium, commonly 4 to 6; typically one flower at the beginning of the season.
   Pedicel hairs.—Pubescence generally disposed somewhat upwards.
   Pedicel color.—Commonly near Yellow-Green Group 144C.
   Size.—Large, with primary flowers approximately 2.5 cm to 3.5 cm in diameter on average, and secondary flowers approximately 2.1 cm to 2.6 cm in diameter on average, commonly the open calyx is larger than the diameter than the corolla being approximately 2.6 cm to 4.5 cm in diameter on average.
   Petals.—Number approximately 5 to 7 on average, typically 6. — arrangement and shape: overlapping, somewhat rounded overall in configuration with the relationship of the length to the width being substantially equal, and acute apex. — size: approximately 10 mm on average in length and width on average. — color: commonly near White Group NN155A on the upper surface.
   Anthers.—Number is approximately 21 to 25 on average, commonly disposed below the stamen, and color is commonly near Yellow Group 6A to Yellow Group 7A.
   Pollen.—In abundance.
   Sepals.—Shape: generally lanceolate in configuration; attitude is commonly upwards, and generally extend beyond the petals. — number: approximately 12 to 14 on average. — size: approximately 1.2 cm to 1.6 cm in length on average, and approximately 5 mm to 7 mm in width on average at the broadest point. — color: the upper surface is commonly near Green Group 143A to Green Group 143B and the under surface is commonly near Green Group 141C to Green Group 142B.
Fruit:
   Bearing.—Non-remontant.
   Timing.—Early fruiting commonly with approximately 28 to 34 days from first blooming to first fruit ripening.
   Shape.—Conical, slightly rhomboid, longer than broad, commonly with a medium difference between terminal and the other fruits.
   Size.—Large, with the primary fruit approximately 5.0 cm to 6.5 cm in length on average; approximately 4 cm to 4.5 cm in width on average at the broadest point; and approximately 23.4 grams on average.
   Surface.—Slightly uneven texture with strong glossiness.
   External color.—Substantially uniform commonly near Red Group 43A to Red Group 43B.
   Internal color.—Flesh is commonly near Orange-Red Group 33A to Orange-Red Group 33B, and the core is commonly near Orange-Red Group 33A to Orange-Red Group 33B.
   Firmness.—Good firmness.
   Cavity.—Small to medium fruit cavity, as illustrated in FIG. 13.
   Achenes.—Located generally below the fruit surface and cover nearly the entire fruit surface commonly with only a very narrow band (if any) where achenes are absent, and commonly going from near Orange Group 28A to Red Group 43B in coloration, depending of fruit maturity.
   Calyx.—Commonly slightly raised at the point of attachment, the calyx commonly attaches to the fruit with medium to hard adherence, the sepals are disposed generally slightly upwards, calyx typically extends beyond the overlapping petals when open, and the fruit diameter of calyx in relation to diameter of fruit commonly are slightly bigger.
   Peduncle.—Long, approximately 30 cm to 36 cm in length on average for primary fruit, and color is commonly near Yellow-Green Group N144C.
   Pedicel.—Commonly with pubescence extending upwards, and color is commonly near Yellow-Green Group N144C.
Development:
   Fertilization.—Self-fertile.
   Resistance to disease.—During the time of observations and to date, no sensitivities to any disease were observed.

Plants of the ‘NSG 120’ variety have not been observed under all possible environmental conditions to date. Accordingly, it is possible that the phenotypic expression may vary somewhat with changes in light intensity and duration, cultural practices, and other environmental conditions.

### SUPPLEMENTAL COMPARATIVE DATA

Hereafter, additional comparative fruit data is provided for the new variety, the ‘NSG 1117’ variety, the ‘MELISSA’ variety (i.e., the male parent) and the ‘CHARLENE’ variety (i.e., the female variety). The plants were asexually reproduced by the use of stolons in 2017 at Zielona Gora-Ochla, Poland located at 51°848 N-15°447 E, and planted in field on Oct. 15, 2017. All varieties were grown under the cover of plastic tunnels at Nova Siri (MT), Italy located at 40°08′40″ N-16°39′40″ E (10 meters above sea level). The fruit were evaluated and compared on the dates indicated. Average data is as follow:

TABLE 1
Accumulated Production of First Quality Fruit (g/plant)

| variety | February 30^{th} | March 30^{th} | April 30^{th} | May 20^{th} |
|---|---|---|---|---|
| ‘NSG 120’ | 58 | 234 | 435 | 643 |
| ‘NSG 1117’ | 24 | 195 | 405 | 600 |

Datos Nicola + 15% + 30%

TABLE 2
Overall Comparison of Average Fruit Weight
in Nova Siri-Italy on year 2018

| plant | g/fruit |
|---|---|
| ‘NSG 120’ | 23.4 |
| ‘MELISSA’ | 26.0 |
| ‘CHARLENE’ | 22.0 |
| ‘NSG 1117’ | 26.2 |

TABLE 3
Average Fruit Weight on Specified Dates on 2018

| | March 30^{th} | April 30^{th} | May 20^{th} |
|---|---|---|---|
| variety | (grams) | (grams) | (grams) |
| ‘NSG 120’ | 24 | 23.5 | 23 |
| ‘MELISSA’ | 27 | 26 | 22 |
| ‘CHARLENE’ | 24 | 22 | 19 |
| ‘NSG 1117’ | 28 | 24 | 25 |

TABLE 4
Fruit Analysis On May 30, 2018

| | ‘NSG 120’ | ‘NSG 1117’ | ‘CHARLENE’ | ‘MELISSA’ |
|---|---|---|---|---|
| Firmness | 0.70 | 0.77 | 0.74 | 0.75 |
| (average)* | | | | |
| Dry Matter | 9.14 | 8.25 | 8.50 | 8.25 |
| (%)** | | | | |
| pH (to 20°) | 3.40 | 3.60 | 3.80 | 3.93 |
| Acidity as | 0.71 | 0.69 | 0.70 | 0.60 |
| Anhydride | | | | |
| Citric (%) | | | | |
| Soluble Solids | 8.50 | 7.60 | 8.80 | 9.00 |
| (% Brix) | | | | |
| Maturity | 9.93 | 11.01 | 12.57 | 15.00 |
| Index *** | | | | |

*Resistance to penetration measured in kilograms using a Turoni (Italy) pentrometer (20 Kg × 0.01).
**Weight of residue from the titration of the fruit after drying at 103° C. until is achieved a constant weight.
*** Relation between soluble solids and acidity anhydride.

I claim:

1. A new and distinct strawberry plant characterized by the following combination of characteristics:
(a) exhibits a semi-dense, semi-upright growth habit,
(b) displays, on an early basis, white inflorescence at a level generally above the foliage,
(c) commonly displays a calyx that is larger than the diameter of the corolla when open, and,
(d) forms in abundance attractive early-ripening large, medium red primarily conical fruit having firm flesh that is longer than broad in configuration;
substantially as herein shown and described.
